(19) United States
(12) Patent Application Publication
(10) Pub. No.: US 2026/0102184 A1
(43) Pub. Date: Apr. 16, 2026

(54) TROCAR, TROCAR HOLDER, AND TROCAR DETECTION SYSTEM FOR SURGICAL ROBOT INCLUDING THE SAME

(71) Applicant: LIVSMED INC., Seongnam-si (KR)

(72) Inventors: Young Jae SONG, Seongnam-si (KR); Se Ra KIM, Suwon-si (KR); So Yeon KIM, Yongin-si (KR); Young Wan KIM, Seongnam-si (KR); Jin Ah SIM, Yongin-si (KR)

(21) Appl. No.: 19/358,845

(22) Filed: Oct. 15, 2025

(30) Foreign Application Priority Data
Oct. 16, 2024 (KR) 10-2024-0141397

(51) Int. Cl. A61B 17/34; A61B 17/00; A61B 34/30; A61B 34/37; A61B 90/50; A61B 90/90

(52) U.S. Cl. CPC A61B 17/3423; A61B 34/37; A61B 90/50; A61B 90/90; A61B 2017/00482; A61B 2034/302

(57) ABSTRACT

A trocar, a trocar holder, and a trocar detection system for a surgical robot including the trocar and the trocar holder are provided. The trocar detection system for a surgical robot including an arm unit includes a trocar holder positioned at one end portion of the arm unit and including a trocar detection unit having a plurality of switches configured to be activated by an external pressure or contact, and a trocar including a mounting part, which is a region configured to be mounted on the trocar holder, wherein the mounting part includes an identification portion having a protruding shape so as to be in physical contact with at least one of the plurality of switches when the trocar is mounted on the trocar holder.

### CROSS-REFERENCE TO RELATED APPLICATION

This application is based on and claims priority under 35 USC § 119 to Korean Patent Application No. 10-2024-0141397, filed on Oct. 16, 2024, in the Korean Intellectual Property Office, the disclosure of which is incorporated by reference herein in its entirety.

### BACKGROUND

### 1. Field

The present disclosure relates to a trocar, a trocar holder, and a trocar detection system for a surgical robot including the same.

### 2. Description of the Related Art

Medically, surgery refers to the treatment of diseases by cutting, slitting, or manipulating the skin, mucous membranes, or other tissues using medical devices. In particular, open surgery, which cuts and opens the skin of a surgical site and cures, shapes, or removes an organ therein, may cause bleeding, side effects, patient pain, scars, or the like. Accordingly, recently, surgery performed by inserting only a medical device, for example, laparoscopic surgical instrument, microsurgical microscope, and the like by forming a predetermined hole in the skin or surgery using a robot has been spotlighted as an alternative.

Here, a surgical robot refers to a robot that has the function of replacing surgical actions performed by a surgeon. Compared to humans, the surgical robot has the advantage of being able to operate with greater accuracy and precision, as well as being able to operate remotely.

Surgical robots that are currently being developed worldwide may include a bone surgical robot, a laparoscopic surgical robot, a stereotactic surgical robot, and the like. Here, the laparoscopic surgical robot is a robot that performs minimum invasive surgery using a laparoscope and small surgical instruments.

Meanwhile, a surgical robot is generally composed of a master robot and a slave robot. When a surgical operator manipulates a control lever (e.g., a handle) equipped on the master robot, a surgical tool coupled to or held by a robot arm equipped on the slave robot may be manipulated to perform surgery.

Laparoscopic surgery is a cutting-edge surgery technique that involves perforating a small hole in the abdomen and inserting a laparoscope, which is an endoscope for looking inside the abdomen to perform the surgery, and is a field that is expected to advance in the future. Today's laparoscopes are mounted with computer chips and have been developed to the extent that magnified images, which are clearer than images seen with the naked eye, can be obtained and when used with specially-designed laparoscopic surgical tools while looking at a monitor screen, any type of surgery is possible.

Meanwhile, in laparoscopic surgery, a surgical instrument called a trocar is used to create a passage into the patient's abdominal cavity, through which surgical instruments such as a laparoscope and other surgical tools are inserted into a surgical site within the cavity, thereby allowing a surgical operator to observe and perform procedures on an internal surgical site.

The background art described above is technical information retained by the present inventors in order to derive the present disclosure or obtained by the present inventors in the process of deriving the present disclosure, and thus is not necessarily known art disclosed to the general public before the filing of the present application.

### SUMMARY

The present disclosure is directed to providing a trocar, a trocar holder, and a trocar detection system for a surgical robot including the same, wherein the trocar detection system for a surgical robot is configured to reduce errors in identifying different types of trocars and to lower manufacturing costs.

According to an aspect of the present disclosure, there is provided a trocar detection system for a surgical robot including an arm unit, the trocar detection system including a trocar holder positioned at one end portion of the arm unit and including a trocar detection unit having a plurality of switches configured to be activated by an external pressure or contact, and a trocar including a mounting part, which is a region configured to be mounted on the trocar holder, wherein the mounting part includes an identification portion having a protruding shape so as to be in physical contact with at least one of the plurality of switches when the trocar is mounted on the trocar holder.

In an embodiment of the present disclosure, the trocar detection unit may generate a unique signal corresponding to a combination of activated switches among the plurality of switches, with respect to the trocar mounted on the trocar holder.

In an embodiment of the present disclosure, the trocar detection system may further include a storage part in which trocar type information that matches the unique signal generated by the trocar detection unit is stored, and a control part configured to determine a type of the trocar based on the trocar type information stored in the storage part and the unique signal transmitted from the trocar detection unit.

In an embodiment of the present disclosure, the identification portion may be formed in a band shape protruding along a circumference of the mounting part.

In an embodiment of the present disclosure, the identification portion may be positioned on an imaginary plane intersecting a central axis of the trocar.

In an embodiment of the present disclosure, the mounting part may have a plurality of identification portions positioned in parallel to each other in a longitudinal direction of the trocar.

In an embodiment of the present disclosure, the trocar may include a first type of trocar and a second type of trocar that are different from each other,
the first type of trocar may include an identification portion that is distinguishable from an identification portion of the second type of trocar, and
a combination of switches activated by the identification portion of the first type of trocar may be different from a combination of switches activated by the identification portion of the second type of trocar, among the plurality of switches.

In an embodiment of the present disclosure, the first type of trocar and the second type of trocar are different from each other in at least one of a number, positions, and shapes of the identification portions.

In an embodiment of the present disclosure, each of the plurality of switches may be positioned within a grid-like section divided into rows and columns, wherein the columns may correspond to a direction parallel to a central axis of the trocar while the trocar is mounted on the trocar holder, and the rows may correspond to a direction orthogonal to the columns, and the plurality of switches may be positioned in the rows, respectively.

In an embodiment of the present disclosure, the plurality of switches may be positioned in two or more of the columns in one of the rows.

In an embodiment of the present disclosure, the plurality of switches are pressure-activated.

In an embodiment of the present disclosure, the mounting part may further include a peripheral portion that does not protrude relative to the identification portion, and, when the trocar is mounted on the trocar holder, the identification portion may press the plurality of switches, and the peripheral portion may not press the plurality of switches.

In an embodiment of the present disclosure, in the mounting part, the identification portion or the peripheral portion may be formed at positions respectively corresponding to the plurality of switches.

In an embodiment of the present disclosure, the trocar holder may further include a pair of grip parts configured to grip the mounting part, and the trocar detection unit may be positioned between the pair of grip parts.

In an embodiment of the present disclosure, a height of the mounting part and a height of each of the pair of grip parts may be defined by a direction parallel to a longitudinal direction of the trocar, and the height of the mounting part may correspond to the height of each of the pair of grip parts.

In an embodiment of the present disclosure, the mounting part may further include an upper boundary portion and a lower boundary portion, wherein the upper boundary portion and the lower boundary portion may guide the pair of grip parts to grip a certain position of the mounting part.

According to another aspect of the present disclosure, there is provided a trocar holder provided on a surgical robot, the trocar holder including a grip part configured to grip a trocar, and a trocar detection unit including a plurality of switches configured to be activated when in contact with or pressed by a protruding shape of the trocar, wherein the trocar detection unit has a combination of activated switches that varies depending on a type of a trocar mounted on the trocar holder.

In an embodiment of the present disclosure, the trocar detection unit may generate a unique signal corresponding to the combination of the activated switches among the plurality of switches, with respect to the trocar mounted on the trocar holder.

According to another aspect of the present disclosure, there is provided a trocar configured to be mounted on a trocar holder including a trocar detection unit having a plurality of switches, the trocar including a cannula having a hollow portion formed therein to allow an insertion of a surgical instrument through the hollow portion, a mounting part disposed at one end portion of the cannula and serving as a region mounted on the trocar holder, wherein the mounting part includes an identification portion having a protruding shape so as to be in physical contact with at least one of the plurality of switches when the trocar is mounted on the trocar holder.

In an embodiment of the present disclosure, the identification portion may be formed in a band shape protruding along a circumference of the mounting part.

Other aspects, features, and advantages other than those described above will become apparent from the following drawings, claims, and detailed description of the disclosure.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a block diagram schematically illustrating a trocar detection system for a surgical robot according to an embodiment of the present disclosure;

FIG. 2 is a conceptual diagram illustrating a surgical robot system including the surgical robot according to an embodiment of the present disclosure;

FIG. 3 is a perspective view of the surgical robot of FIG. 2;

FIG. 4 is a magnified perspective view illustrating some components of a first arm unit of FIG. 3;

FIG. 5 is a magnified perspective view of a trocar holder and a trocar of FIG. 4;

FIG. 6 is a perspective view of the trocar holder and the trocar of FIG. 5, shown from a different angle;

FIG. 7 is a perspective view illustrating the trocar of FIG. 6 in a state separated from the trocar holder;

FIG. 8 is a perspective view illustrating the trocar and a trocar detection unit of FIG. 7;

FIG. 9 is a cross-sectional view illustrating cross sections of the trocar and the trocar holder of FIG. 7;

FIG. 10 is a perspective view illustrating a trocar detection unit according to another embodiment of the present disclosure;

FIG. 11 is a perspective view illustrating a trocar and a trocar holder according to another embodiment of the present disclosure;

FIG. 12 is a cross-sectional view illustrating cross sections of the trocar and the trocar holder of FIG. 11; and

FIG. 13 is a cross-sectional view illustrating a cross section of a trocar according to another embodiment of the present disclosure.

### DETAILED DESCRIPTION

Hereinafter, the following embodiments will be described in detail with reference to the accompanying drawings. When describing with reference to the drawings, identical or corresponding components will be assigned the same reference numerals and duplicate descriptions thereof will be omitted.

Since various transformations can be made to these embodiments, specific embodiments will be illustrated in the drawings and described in detail in the detailed description. The effects and features of the present embodiments and the accompanying methods thereof will become apparent from the following description of the contents, taken in conjunction with the accompanying drawings. However, the present embodiments are not limited to the embodiments disclosed below, but may be implemented in various forms.

In describing the present disclosure, a detailed description of known related arts will be omitted when it is determined that the gist of the present disclosure may be unnecessarily obscured.

In the following embodiments, singular forms are intended to include plural forms as well, unless the context clearly indicates otherwise. Although terms such as “first,” “second,” and the like may be used to describe various components, such components should not be limited to the above terms. The terms are only used to distinguish one component from another.

In the following embodiments, terms such as “include” or “have” means that the features or components described in the specification are present, and the possibility that one or more other features or components will be added is not excluded in advance.

In the following embodiments, when a unit, region, or component is referred to as being formed on another unit, region, or component, it can be directly formed on the other unit, region, or component. That is, for example, intervening units, regions, or components may be present.

In the following embodiments, terms such as “connecting” or “coupling” two members do not necessarily mean a direct and/or fixed connection or coupling of the two members, unless the context clearly indicates otherwise, and do not preclude another member from being interposed between the two members.

Sizes of components in the drawings may be exaggerated or reduced for convenience of description. For example, since the size and thickness of each component shown in the drawings are arbitrarily illustrated for convenience of description, the following embodiments are not necessarily limited thereto.

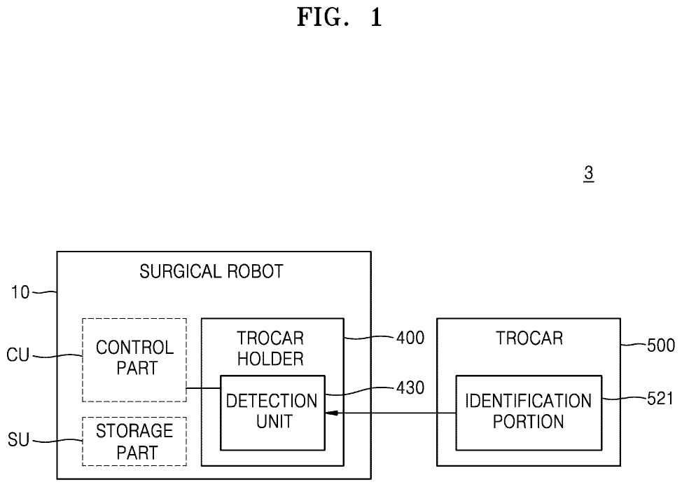

FIG. 1 is a block diagram schematically illustrating a trocar detection system for a surgical robot according to an embodiment of the present disclosure.

Referring to FIG. 1, a trocar detection system 3 for a surgical robot 10 according to an embodiment of the present disclosure may include the surgical robot 10 and a trocar 500.

The surgical robot 10 may include a trocar holder 400 in which a trocar 500 may be mounted. In some embodiments, the trocar holder 400 may include a trocar detection unit 430.

Here, the trocar detection unit 430 may identify a type of the trocar 500 being mounted. The surgical robot 10 may include at least two types of trocars 500 that may be mounted on the trocar holder 400. The trocar detection unit 430 may recognize the type of the trocar 500 mounted on the trocar holder 400, and the surgical robot 10 may control operations of an arm unit based on recognized data to perform an appropriate surgical operation.

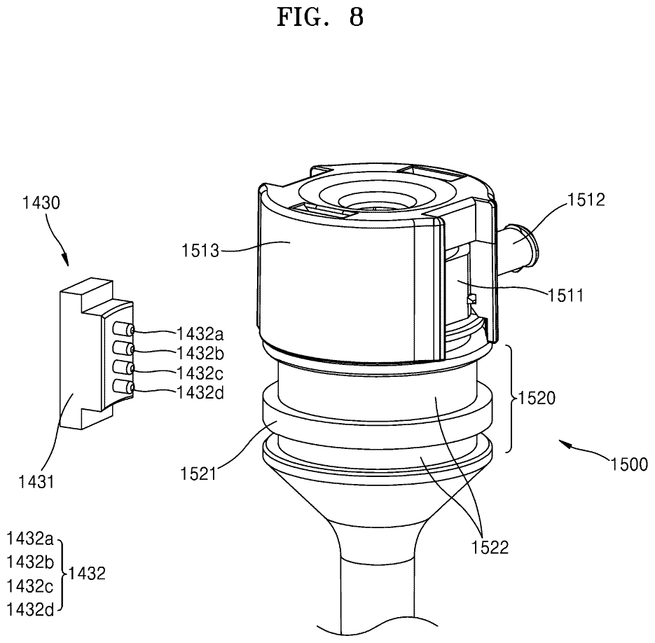

In some embodiments, the trocar detection unit 430 may include a plurality of switches 1432a to 1432d (see FIG. 8) that are activated by external pressing or contact. In some embodiments, the trocar detection unit 430 may generate a unique signal corresponding to the trocar 500 mounted on the trocar holder 400, based on a combination of activated switches among the plurality of switches.

In some embodiments, the trocar 500 may include an identification portion 521 configured to provide an input signal to the trocar detection unit 430 when mounted on the trocar holder 400.

For example, the identification portion 521 of the trocar 500 may come into physical contact with or press against the trocar detection unit 430 to activate a specific switch.

In some embodiments, the identification portion 521 may be a protrusion formed in one region of the trocar 500. In some embodiments, the identification portion 521 may be a protrusion shaped to come into physical contact with at least one of the plurality of switches. In some embodiments, the shape of the protrusion may be formed differently for each type of the trocar 500.

Accordingly, a particular trocar may include the identification portion 521 of a specific shape to activate a specific switch of the trocar detection unit 430. As such, each type of trocar may include a different identification portion 521, and depending on the identification portion 521, the trocar detection unit 430 may generate a different unique signal.

In some embodiments, the surgical robot 10 may further include a storage part SU and a control part CU. Here, the storage part SU may store trocar type information regarding the type of the trocar 500. That is, information previously entered about the trocar 500 that is mountable to the trocar holder 400 may be stored in the storage part SU.

For example, the trocar type information may be information about the trocar 500 that matches the unique signal generated by the trocar detection unit 430.

The control part CU may determine the type of the trocar 500 based on the trocar type information stored in the storage part SU and the unique signal transmitted from the trocar detection unit 430.

In some embodiments, the control part CU may compare data corresponding to the unique signal transmitted from the trocar detection unit 430 with data stored in the storage part SU and extract trocar type information matching the comparison result.

Accordingly, the trocar detection system 3 may identify the mounted trocar 500 based on the unique signal generated according to the combination of switches activated by the identification portion 521, when the trocar 500 including the identification portion 521 is mounted on the trocar holder 400 including the trocar detection unit 430.

The trocar detection unit 430 and the identification portion 521 will be described in detail later.

Hereinafter, the surgical robot 10 according to an embodiment of the present disclosure will be described.

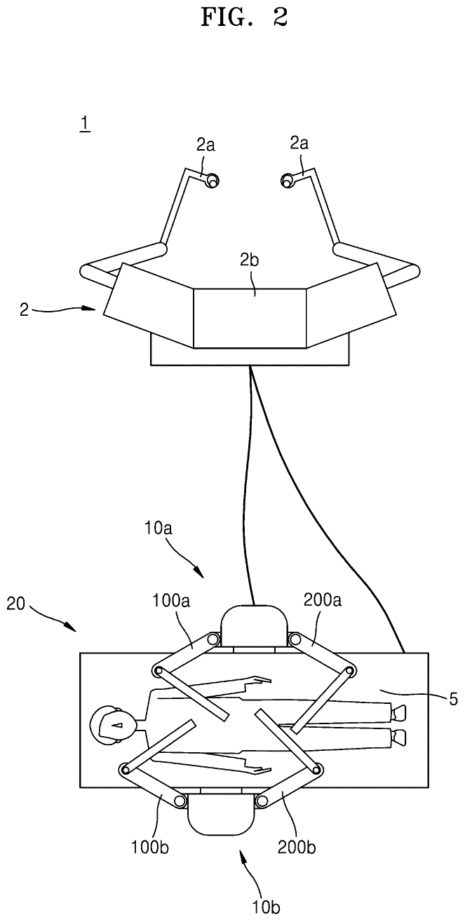

FIG. 2 is a conceptual diagram illustrating a surgical robot system including the surgical robot according to an embodiment of the present disclosure.

Referring to FIG. 2, a surgical robot system 1 includes a master robot 2 and the surgical robot 10.

The master robot 2 includes manipulating members and a display member, and the surgical robot 10 includes one or more robot arm units 100 and 200.

In some embodiments, the master robot 2 includes manipulating members 2a so that a surgical operator can grip and manipulate them respectively with both hands. In some embodiments, an image captured through a laparoscope is displayed as a screen image on a display member 2b of the master robot 2. In some embodiments, a predetermined virtual manipulation plate may be displayed independently or displayed together with the image captured by the laparoscope or the like on the display member 2b. A detailed description of the arrangement, configuration, and the like of such a virtual manipulation plate will be omitted.

In some embodiments, the surgical robot 10 may include at least two robot arm units 100 and 200. Here, each of the robot arm units 100 and 200 may be provided in the form of a module that can operate independently of each other, and in this case, an algorithm for preventing a collision between the robot arm units 100 and 200 may be applied to the surgical robot system 1.

The surgical robot system 1 may include one or more surgical robots 10. FIG. 2 illustrates an embodiment in which the surgical robot system 1 includes two surgical robots 10a and 10b, each having two robot arm units 100 and 200, such that a total of four robot arm units 100a, 200a, 100b, and 200b are disposed.

In an embodiment, two or more of the robot arm units 100 and 200 may each have a surgical instrument SI attached thereto, and one or more of the robot arm units 100 and 200 may have a laparoscope attached thereto. In some embodiments, the surgical operator may select any of the robot arm units 100a, 200a, 100b, and 200b to be controlled through the master robot 2. As described above, by directly controlling three or more surgical instruments through the master robot 2, the surgical operator may accurately and freely control various tools on the surgical bed 5 as intended, without the need for a surgical assistant.

Hereinafter, the configuration and operating principle of the surgical robot 10 will be described in detail.

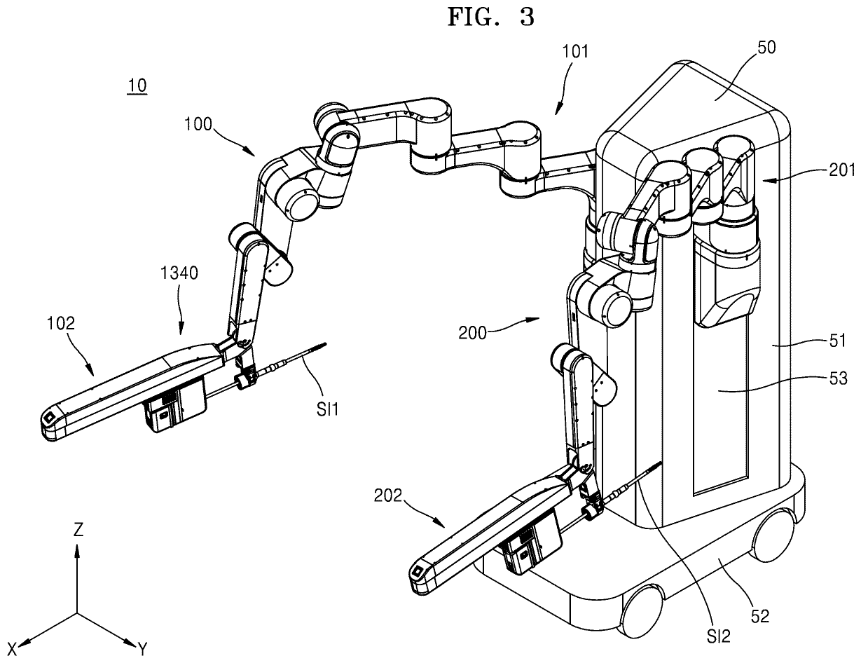

FIG. 3 is a perspective view of the surgical robot of FIG. 2.

Referring to FIG. 3, the surgical robot 10 may include a body part 50, a first arm unit 100, and a second arm unit 200. FIG. 3 illustrates an embodiment in which the surgical robot 10 includes two robot arm units, and hereinafter, the robot arm units are defined as the first arm unit 100 and the second arm unit 200, respectively.

The body part 50 functions as a body of the surgical robot 10, on which the first arm unit 100 and the second arm unit 200 may be disposed. In some embodiments, the body part 50 may serve as a reference point for driving the first arm unit 100 and the second arm unit 200.

The body part 50 may include a first body 51 and a second body 52. The first arm unit 100 and the second arm unit 200 may be disposed on the first body 51, and the second body 52 may support the first body 51. In some embodiments, the second body 52 may have wheels, as shown in FIG. 3, thereby enabling the surgical robot 10 to move.

The body part 50 may have a lifting guide 53. The lifting guide 53 may be provided corresponding to the number of robot arm units disposed on the body part 50. The lifting guide 53 may be recessed into one side of the body part 50, and each of the robot arm units 100 and 200 may be coupled to the lifting guide 53 to be slidable in a first direction.

In describing the present disclosure, the portion closer to the body part 50 will be referred to as a proximal end, and the portion farther from the body part 50 will be referred to as a distal end. For example, the portion of the first arm unit 100 closer to the body part 50 is defined and described as a proximal end 101 of the first arm unit 100, and the portion of the first arm unit 100 farther from the body part 50 is defined and described as a distal end 102 of the first arm unit 100. In some embodiments, the portion of the second arm unit 200 closer to the body part 50 is defined and described as a proximal end 201 of the second arm unit 200, and the portion of the second arm unit 200 farther from the body part 50 is defined and described as a distal end 202 of the second arm unit 200.

The first arm unit 100 may be disposed on one side of the body part 50 and may have a first surgical instrument SI1 mounted thereon. The surgical robot 10 may drive the first arm unit 100 to adjust the position and posture of the first surgical instrument SI1.

A first arm connection part (not shown) may include a plurality of connection links, and the posture may be determined according to driving of each connection link. A remote center of motion (RCM) point RCM1 of the first surgical instrument SI1 may be determined according to the posture of the first arm connection part. In some embodiments, the RCM point of the first surgical instrument SI1 refers to an imaginary center point that serves as the reference for the rotation of the first surgical instrument SI1. The first surgical instrument SI1 rotates around the RCM point and may perform yaw and pitch motions.

The second arm unit 200 may be disposed on another side of the body part 50 and may have a second surgical instrument SI2 mounted thereon. The surgical robot 10 may drive the second arm unit 200 to adjust the position and posture of the second surgical instrument SI2.

A second arm connection part (not shown) may include a plurality of connection links, and a posture thereof may be determined according to driving of each connection link. An RCM point RCM1 of the second surgical instrument SI2 may be determined according to the posture of the second arm connection part. In some embodiments, the RCM point of the second surgical instrument SI2 refers to an imaginary center point that serves as the reference for the rotation of the second surgical instrument SI2. The second surgical instrument SI2 rotates around the RCM point and may perform yaw and pitch motions.

Each of the arm units may include a plurality of arm connection links and a plurality of arm extension links. The arm connection links and the arm extension links may rotate around their respective reference axes, and through these rotational motions, the arm unit may adjust the posture and placement of the surgical instrument within the operational range.

Here, a detailed description of the arm connection links and arm extension links will be omitted.

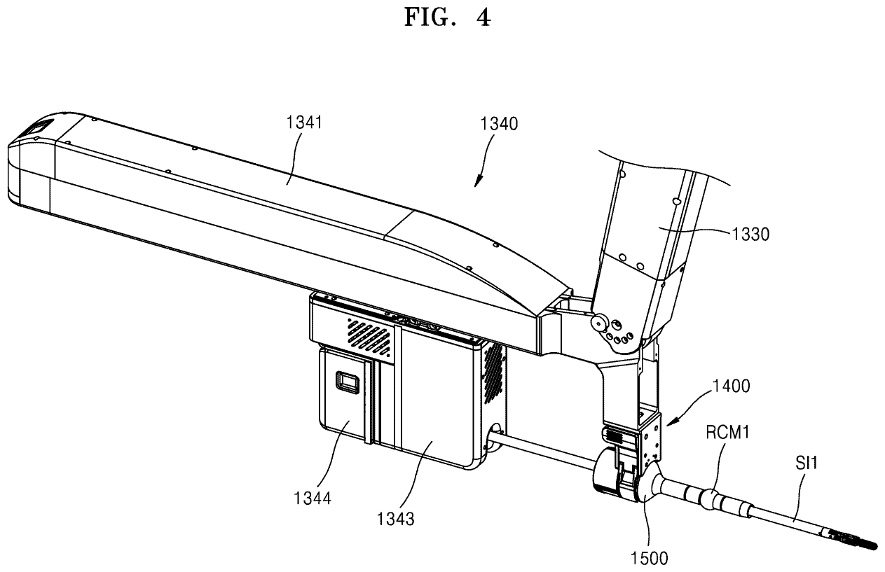

FIG. 4 is a magnified perspective view illustrating some components of the first arm unit 100 of FIG. 3.

Referring to FIGS. 3 and 4, the first arm unit 100 according to an embodiment may include a first arm slide link 1340 on the distal end 102 side.

Here, the first arm slide link 1340 may allow sliding movement of the first surgical instrument SI1.

The first arm slide link 1340 may be coupled to one end of a first arm third extension link 1330, i.e., on the distal end side, and the first surgical instrument SI1 may be disposed on the first arm slide link 1340.

The first arm slide link 1340 may include a translation arm 1341, a slide motor pack 1343, a slide driving part 1344, and a trocar holder 1400.

The translation arm 1341 is connected to one end of the first arm third extension link 1330 and may move together with the first arm third extension link 1330. That is, when the first arm third extension link 1330 is driven, a posture of the translation arm 1341 may change accordingly.

The slide motor pack 1343 may provide a driving force for the sliding movement of the first surgical instrument SI1. The slide motor pack 1343 may include one or more first motors, as well as various components capable of generating and transmitting a driving force.

The slide driving part 1344 may receive a driving force from the slide motor pack 1343 and move the first surgical instrument SI1 in a sliding manner. One end of the first surgical instrument SI1 is connected to the slide driving part 1344, and the first surgical instrument SI1 may be linearly moved by the slide driving part 1344.

The trocar holder 1400 is disposed in one region of the translation arm 1341, and a trocar 1500 may be mounted on the trocar holder 1400. In some embodiments, the trocar holder 1400 may be positioned close to an end portion of the first arm third extension link 1330 on the distal end side.

The trocar 1500 is mountable to the trocar holder 1400, and the first surgical instrument SI1 may be coupled to the trocar 1500. The first surgical instrument SI1 may be mounted through the trocar 1500. In some embodiments, the first surgical instrument SI1 is partially supported on the trocar 1500 and may be slidably movable.

The RCM point RCM1 of the first surgical instrument SI1 may be formed on one side of the trocar 1500. That is, the trocar 1500 may provide an RCM point, which serves as a reference point for rotational movements, including yaw and pitch motions, of the first surgical instrument SI1 on one side. Once the posture of the first arm connection part is determined, a position of the RCM point on the trocar 1500 is also determined, and even when the first surgical instrument SI1 slidably moves, the position of the RCM point may remain fixed.

Hereinafter, the trocar holder 1400 and the trocar 1500 according to an embodiment of the present disclosure will be described in detail.

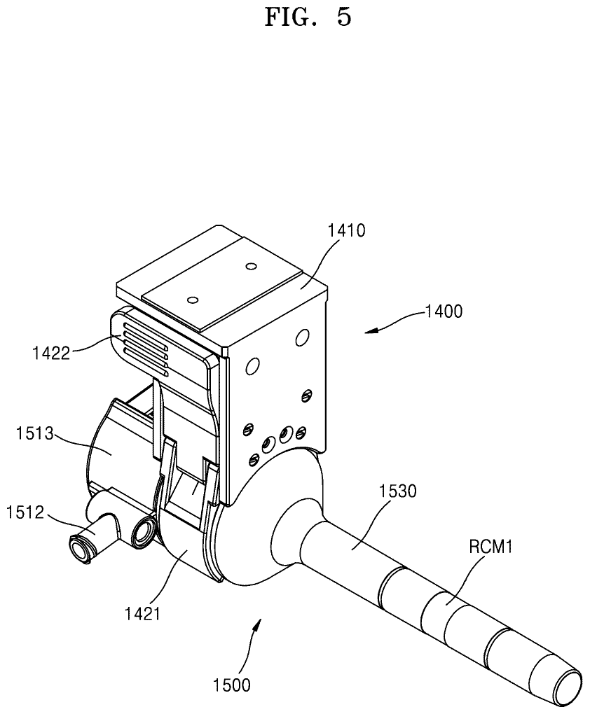

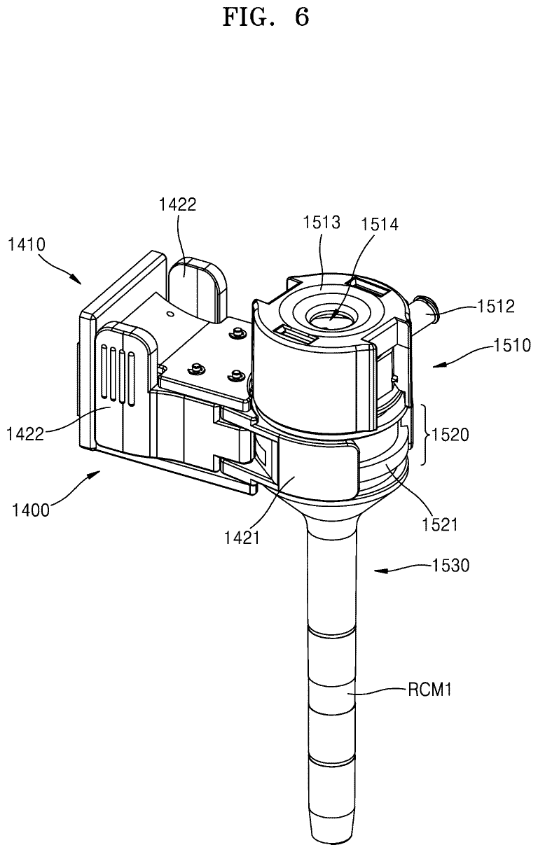

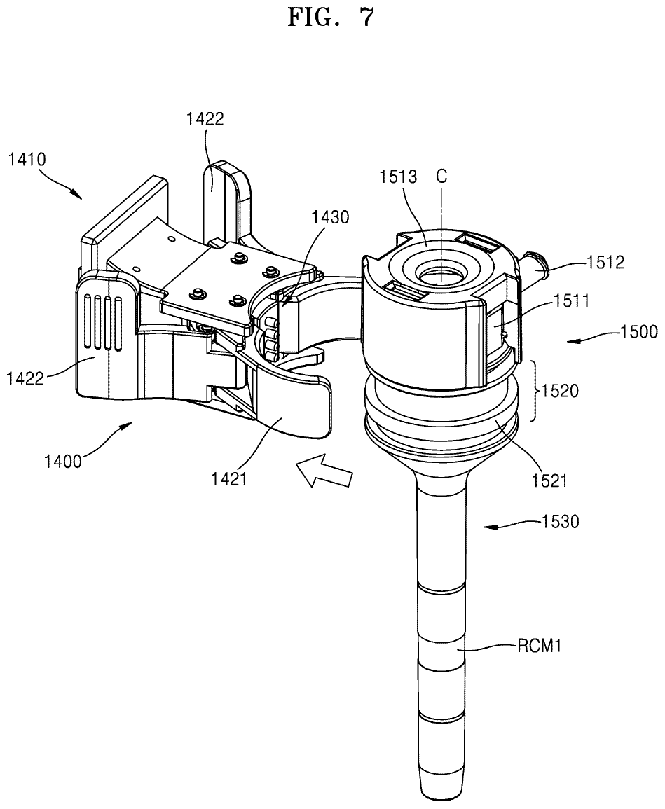

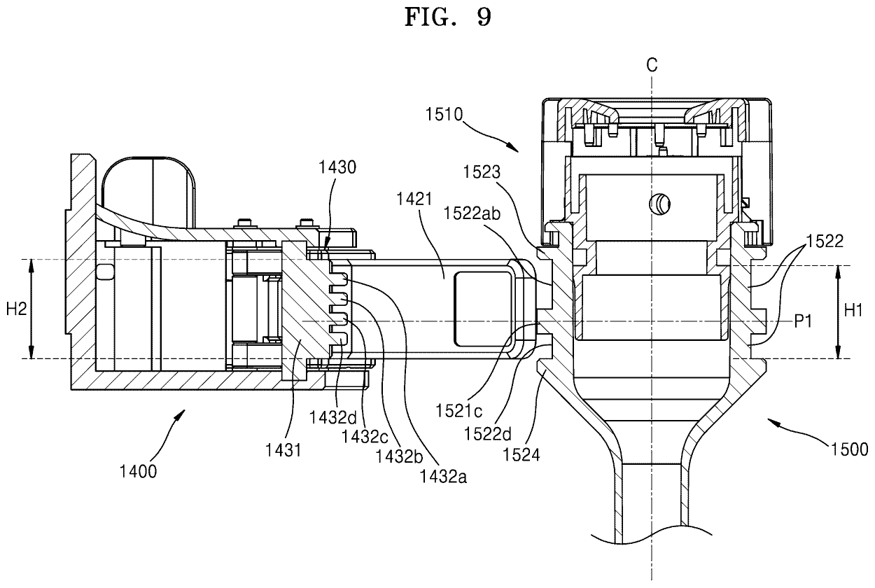

FIG. 5 is a magnified perspective view of the trocar holder and the trocar of FIG. 4, and FIG. 6 is a perspective view of the trocar holder and the trocar of FIG. 5, shown from a different angle. FIG. 7 is a perspective view illustrating the trocar of FIG. 6 in a state separated from the trocar holder, FIG. 8 is a perspective view illustrating the trocar and the trocar detection unit of FIG. 7, and FIG. 9 is a cross-sectional view illustrating cross sections of the trocar and the trocar holder of FIG. 7.

Referring to FIGS. 5 to 9, the trocar holder 1400 according to an embodiment of the present disclosure may include a holder body part 1410, a grip part 1421, a pressing part 1422, and a trocar detection unit 1430.

The holder body part 1410 may accommodate at least some of the components, such as the grip part 1421, the pressing part 1422, and the trocar detection unit 1430 to be described later. In some embodiments, the holder body part 1410 may accommodate electronic components and conducting wires, which are connected to the trocar detection unit 1430, therein. One end portion of the holder body part 1410 may be coupled to the translation arm 1341 described above.

The grip part 1421 may be positioned at another end portion of the holder body part 1410. One end portion of the grip part 1421 may be connected to the holder body part 1410 to be rotatable around one axis. In some embodiments, another end portion of the grip part 1421 may extend in a direction away from the holder body part 1410.

In some embodiments, the trocar holder 1400 may include a pair of grip parts 1421. That is, the pair of grip parts 1421 are positioned to face each other and may be rotatable toward or away from each other. In this case, the pair of grip parts 1421 may grip the trocar 1500 when rotated toward each other. In some embodiments, the pair of grip parts 1421 may release the grip on the trocar 1500 when rotated away from each other. That is, the trocar 1500 may be detachable from the trocar holder 1400.

The pressing parts 1422 may be positioned on one side surface or opposite side surfaces of the holder body part 1410. The pressing parts 1422 are members that are rotatable around one axis, and one end portion of each of the pressing parts 1422 is spaced apart from the holder body part 1410. When one end portion of each of the pressing parts 1422 rotates in a direction closer to the holder body part 1410, the grip parts 1421 may perform a closing motion.

That is, when a user presses the pressing parts 1422 positioned on opposite side surfaces of the holder body part 1410, the grip parts 1421 may perform a gripping motion.

In some embodiments, the trocar detection unit 1430 may be positioned at another end portion of the holder body part 1410. In some embodiments, the trocar detection unit 1430 may be positioned between the pair of grip parts 1421.

That is, the trocar detection unit 1430 may be positioned between a rotation axis that becomes the center of rotation of one grip part 1421 and a rotation axis that becomes the center of rotation of another grip part 1421.

However, the position in which the trocar detection unit 1430 is disposed is not limited thereto and may be set to any position where the mounting part 1520 of the trocar 1500 can come into contact when the trocar 1500 is mounted on the trocar holder 1400.

The trocar detection unit 1430 may include a housing 1431 and a switch unit 1432. The housing 1431 is the part in which the switch unit 1432 is positioned and may be the part that is fastened to the holder body part 1410. The switch unit 1432 may include a plurality of switches. In some embodiments, the plurality of switches may be collectively referred to as the switch unit 1432.

As described above, the trocar detection unit 1430 may include a plurality of switches that are activated in response to external pressing or contact.

For example, the plurality of switches may be pressure-activated switches, such as pogo pin switches. In some embodiments, the plurality of switches may be pressure-activated switches, in which protruding parts, such as plungers, are inserted into barrels by external pressure to form electrical contacts.

In some embodiments, the plurality of switches may be sensors including pressure conductive rubber. However, the types and methods of the switches are not limited thereto.

In an embodiment, the plurality of switches may be disposed in a plurality of rows that are distinguished in a direction parallel to a central axis C of the trocar 1500 when the trocar 1500 is mounted on the trocar holder 1400.

In other words, the plurality of switches may be disposed in a direction parallel to a longitudinal direction of the trocar 1500.

For example, the plurality of switches may be arranged in a line. In some embodiments, referring to FIG. 8, the trocar detection unit 1430 may include a first switch 1432a, a second switch 1432b, a third switch 1432c, and a fourth switch 1432d, and the first to fourth switches 1432a to 1432d may be arranged in a line.

In another embodiment, the plurality of switches may be arranged in different rows, but may not be arranged in a line. That is, the first switch 1432a may be disposed in a first row, a second switch 1432b may be disposed in a second row, a third switch 1432c may be positioned in a third row, and a fourth switch 1432d may be disposed in a fourth row, but, the first to fourth switches 1432a to 1432d may not be arranged in a direction parallel to the longitudinal direction of the trocar 1500.

In some embodiments, an operating state of the switch may be represented as ON/OFF. That is, one switch may generate two types of input signals.

For example, the switch unit 1432 consisting of four switches may generate 16 types of input signals, whereas the switch unit 1432 consisting of five switches may generate 32 types of input signals.

As described above, when a different switch among the plurality of switches is activated, the input signal may also change.

In other words, the trocar detection unit 1430 may generate a unique signal corresponding to a combination of activated switches among the plurality of switches.

The trocar detection system 3 according to an embodiment of the present disclosure may determine the type of the trocar 1500 using the unique signal generated by the trocar detection unit 1430.

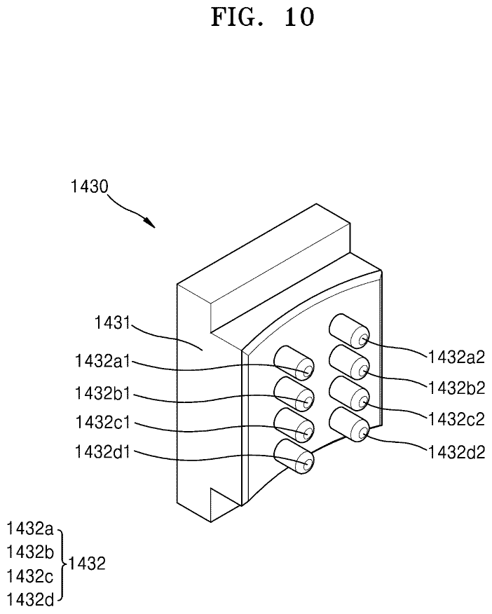

FIG. 10 is a perspective view illustrating a trocar detection unit 1430 according to another embodiment of the present disclosure.

Referring to FIG. 10, a plurality of switches may be arranged in at least two columns in one row.

For example, a first-1 switch 1432a1 and a first-2 switch 1432a2 may be disposed in a first row, a second-1 switch 1432b1 and a second-2 switch 1432b2 may be disposed in a second row, a third-1 switch 1432c1 and a third-2 switch 1432c2 may be disposed in a third row, and a fourth-1 switch 1432d1 and a fourth-2 switch 1432d2 may be disposed in a fourth row.

Here, the switches disposed in the same row may ultimately function as a single switch. In some embodiments, when only one of the first-1 switch 1432a1 or the first-2 switch 1432a2 is activated, or when neither of them is activated, an input signal corresponding to the first row is generated as an OFF signal, and when both the first-1 switch 1432a1 and the first-2 switch 1432a2 are activated, the input signal corresponding to the first row is generated as an ON signal.

In other words, even when a switch unit 1432 according to the present embodiment includes eight switches, the switch unit 1432 may generate 16 types of input signals as if one switch is disposed in each of the four rows.

That is, the trocar detection unit 1430 may generate a normal input signal only when two switches disposed in one row are pressed together.

Accordingly, since the trocar 1500 must be correctly mounted on a trocar holder 1400 so that an identification portion 1521 of the trocar 1500 can accurately press each switch, the trocar detection unit 1430 according to the present embodiment can account for cases where the switches are improperly pressed.

However, the number and arrangement of the switches in the trocar detection unit 1430 are not limited thereto, and it is of course possible to have a trocar detection unit 1430 with a different number and arrangement of switches depending on the type of the trocar 1500 to be detected by the trocar holder 1400.

Hereinafter, the trocar 1500 according to an embodiment of the present disclosure will be described.

Referring again to FIGS. 5 to 9, the trocar 1500 according to an embodiment of the present disclosure may include the mounting part 1520, an upper module 1510, and a cannula 1530.

The upper module 1510 is a structure in which a hollow portion is formed to allow a surgical instrument to be inserted therethrough. In some embodiments, various components may be positioned inside the upper module 1510 to enable the trocar 1500 to perform its functions.

The upper module 1510 may include an inner case 1511, a gas insertion tube 1512, and a cover part 1513.

The inner case 1511 may be formed in a hollow cylindrical shape and may accommodate components of the upper module 1510 therein. In some embodiments, the gas insertion tube 1512, which will be described later, may be connected to one side surface of the inner case 1511.

The gas insertion tube 1512 may extend in one direction from one side surface of the inner case 1511. Carbon dioxide may be supplied through the gas insertion tube 1512 for injection into the patient's body. The injected carbon dioxide serves to expand the patient's abdominal cavity and create a space for performing laparoscopic surgery. The gas insertion tube 1512 may be made of a material such as plastic or may include a flexible material that undergoes elastic deformation so as to be elastically bendable.

In some embodiments, a gas valve (not shown) may be formed on the gas insertion tube 1512 to control the opening and closing of the gas insertion tube 1512.

In some embodiments, although not shown in the drawing, the inner case 1511 may further include a check valve (not shown). The check valve serves to prevent gas (carbon dioxide) injected into the body through the gas insertion tube 1512 from leaking out of the trocar 1500. The check valve is fixedly coupled inside the inner case 1511 and is configured to open when a surgical instrument is inserted into the body and to close when the surgical instrument is removed from the body, thereby preventing the gas injected into the body from leaking out.

The inner case 1511 may be formed so that at least one region is coupled to the mounting part 1520, which will be described later. In some embodiments, one region of the inner case 1511 may be formed smaller than an inner diameter of the mounting part 1520 and thus may be inserted into the mounting part 1520.

The cover part 1513 is coupled to an upper portion of the inner case 1511 and may serve to assemble and seal the above-described components positioned inside the inner case 1511. In an embodiment, the cover part 1513 may cover a side surface and an upper region of the inner case 1511.

An opening 1514 may be formed in an upper surface of the cover part 1513. In some embodiments, the opening 1514 is formed in a central portion of the cover part 1513, through which a surgical instrument may be inserted. That is, the opening 1514 of the cover part 1513 may serve as an inlet of a communication tube that communicates from the inner case 1511 to a hollow portion of the cannula 1530 to be described later.

In some embodiments, an end tool mounting member (not shown) may be detachably attached to an upper portion of the cover part 1513. For example, a type of groove may be formed in an upper surface of the cover part 1513, such that additional components, such as the end tool mounting member, may be coupled thereto.

The cannula 1530 is coupled to one end portion, i.e., a lower end portion, of the upper module 1510, and has the hollow portion formed therein to allow a surgical instrument to be inserted therethrough. The cannula 1530 is inserted into the patient's body through the navel or a skin incision, and in this state, a surgical instrument that has passed through the upper module 1510 passes through the cannula 1530 to enter the patient's body.

In some embodiments, the above-described RCM point RCM1 may be provided on the cannula 1530. That is, the cannula 1530 may provide the RCM point RCM1, which serves as a reference point for rotation, including yaw and pitch motions of the surgical instrument.

In an embodiment, the cannula 1530 may include the mounting part 1520 to be described later. The mounting part 1520 is a region mounted on the trocar holder 1400 and may be a region formed between the cannula 1530 and the upper module 1510.

In some embodiments, the mounting part 1520 may be formed on an outer circumferential surface of an upper end portion of the cannula 1530. That is, the mounting part 1520 may be formed integrally with the cannula 1530.

In another embodiment, the mounting part 1520 may be a member that is separable from and couplable to the cannula 1530. That is, the mounting part 1520 is a region positioned between the cannula 1530 and the upper module 1510 and may connect the cannula 1530 to the upper module 1510. In some embodiments, the above-described inner case 1511 may be inserted into the mounting part 1520, thereby allowing the inner case 1511 to be coupled to the mounting part 1520.

Hereinafter, the mounting part 1520 and the identification portion 1521 will be described in detail.

As described above, the mounting part 1520 may be a region mounted on the trocar holder 1400. In some embodiments, the mounting part 1520 may be a portion gripped by the grip parts 1421 of the trocar holder 1400.

In some embodiments, the mounting part 1520 may include the identification portion 1521, a peripheral portion 1522, an upper boundary portion 1523, and a lower boundary portion 1524.

Here, the identification portion 1521 may be a protruding region formed on an outer surface of the mounting part 1520. As described above, the identification portion 1521 may be formed in a protruding shape to come into physical contact with the switches provided on the trocar holder 1400.

In some embodiments, the mounting part 1520 may include an identification portion 1521 formed in a protruding shape to come into physical contact with at least one of the plurality of switches when the trocar 1500 is mounted on the trocar holder 1400.

In some embodiments, the identification portion 1521 may be formed in a band shape protruding along a circumference of the mounting part 1520. For example, the mounting part 1520 may have the form of a cylinder, and the identification portion 1521 in a band shape may be formed on an outer circumferential surface of the cylinder. In some embodiments, the identification portion 1521 may be formed with a consistent width along the circumference of the mounting part 1520.

As shown in the diagram, the identification portion 1521 may be in the form of a ring that encircles the outer circumferential surface of the mounting part 1520. However, the concept of the present disclosure is not necessarily limited thereto, and the identification portion 1521 may also be formed in a partial region of the mounting part 1520.

The identification portion 1521 may be formed at a position facing the switches of the trocar holder 1400. For example, the identification portion 1521 may be configured to press one of the plurality of switches while the trocar 1500 is mounted on the trocar holder 1400.

Referring again to FIG. 9, the identification portion 1521 may be positioned on an imaginary plane intersecting the central axis C of the trocar 1500. For example, the identification portion 1521 may be positioned on a first plane P1 perpendicular to the central axis of the trocar 1500.

In other words, the identification portion 1521 is not formed in an inclined spiral shape on the outer surface of the mounting part 1520, and may be positioned at the same height regardless of the direction from which the circumference of the mounting part 1520 is viewed.

Since the identification portion 1521 is formed at the same position on the outer circumferential surface of the mounting part 1520 from all side directions, when the trocar 1500 is mounted on the trocar holder 1400, the identification portion 1521 can be positioned to face the same switch.

In other words, when a direction of an arrow shown in FIG. 7 is assumed to be a direction in which the trocar 1500 is mounted, the identification portion 1521 may press the same switch regardless of which side of the mounting part 1520 faces the mounting direction.

For example, in the embodiment shown in the diagram, when the trocar 1500 is mounted on the trocar holder 1400, the identification portion 1521 may press the third switch 1432c.

In some embodiments, the mounting part 1520 may include the peripheral portion 1522 that does not protrude relative to the identification portion 1521. In other words, the peripheral portion 1522 may be described as a peripheral portion of the identification portion 1521 formed on the outer circumferential surface of the mounting part 1520.

The identification portion 1521 or the peripheral portion 1522 may be formed at positions on the mounting part 1520 corresponding to the plurality of switches.

In some embodiments, when the trocar 1500 is mounted on the trocar holder 1400, the identification portion 1521 may press the switch, and the peripheral portion 1522 may not press the switch.

That is, even when the trocar 1500 is mounted on the trocar holder 1400, the peripheral portion 1522 may be spaced apart from the switch.

Accordingly, the switches activated in the trocar detection unit 1430 may vary depending on how the identification portion 1521 and the peripheral portion 1522 are positioned on the mounting part 1520.

The trocar detection system 3 according to an embodiment of the present disclosure may detect different types of trocars 1500. That is, various types of trocars 1500 may be available for use with surgical robots. For example, the trocars 1500 may be classified based on their shape, such as length and diameter, or according to the type of surgical instrument being inserted.

The trocars 1500 may include a first type of trocar and a second type of trocar that are different from each other. Here, the first type of trocar may include an identification portion that is distinguishable from that of the second type of trocar. In some embodiments, among the plurality of switches in the trocar detection unit 1430, the switch activated by the identification portion of the first type of trocar may be different from the switch activated by the identification portion of the second type of trocar.

That is, the first type of trocar and the second type of trocar may be different from each other in at least one of the number, position, and shape of the identification portions.

Hereinafter, different types of identification portions 1521 of the trocar 1500 will be described by way of example with reference to the drawings.

Referring again to FIG. 9, the trocar 1500 according to an embodiment of the present disclosure may have a first peripheral portion 1522ab formed at positions corresponding to the first switch 1432a and the second switch 1432b, and a first identification portion 1521c formed at a position corresponding to the third switch 1432c. In some embodiments, a second peripheral portion 1522d may be additionally formed at a position corresponding to the fourth switch 1432d.

Thus, when the trocar 1500 according to the present embodiment is mounted on the trocar holder 1400, only the third switch 1432c is activated, and the trocar detection unit 1430 may generate a unique signal corresponding to the activation.

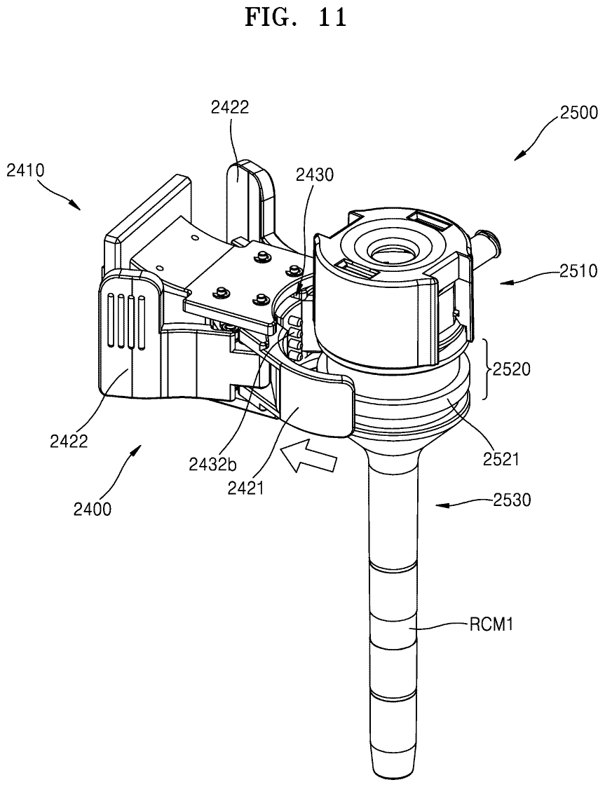

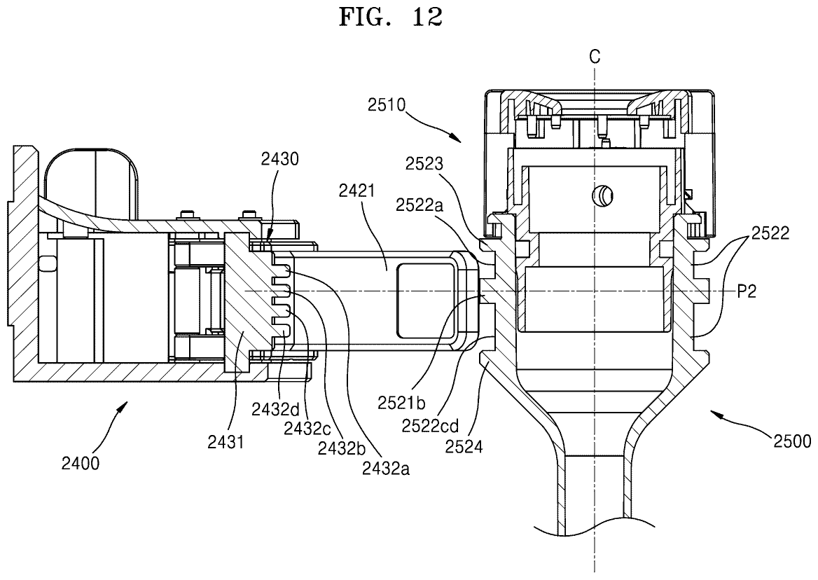

FIG. 11 is a perspective view illustrating a trocar 2500 and a trocar holder 2400 according to another embodiment of the present disclosure, and FIG. 12 is a cross-sectional view illustrating cross sections of the trocar 2500 and the trocar holder 2400 of FIG. 11.

Referring to FIGS. 11 and 12, an identification portion 2521 may be disposed on a second plane P2 perpendicular to a central axis C of the trocar 2500.

The trocar 2500 according to another embodiment of the present disclosure may have a first peripheral portion 2522a formed at a position corresponding to a first switch 2432a, and a first identification portion 2521b formed at a position corresponding to a second switch 2432b. In some embodiments, a second peripheral portion 2522cd may be additionally formed at positions corresponding to a third switch 2432c and a fourth switch 2432d.

Thus, when the trocar 2500 according to the present embodiment is mounted on the trocar holder 2400, only the second switch 2432b is activated and a trocar detection unit 2430 may generate a unique signal corresponding to the activation.

In the present embodiment, the identification portion 2521 is formed to correspond to only one switch, but the concept of the present disclosure is not limited thereto, and one identification portion 2521 may be formed to correspond to two or more switches. For example, one identification portion 2521 may be formed at positions corresponding to the second switch 2432b and the third switch 2432c. That is, a width of the identification portion 2521 may be variously set.

In some embodiments, a mounting part 2520 may include a plurality of identification portions 2521 disposed parallel to each other in a longitudinal direction of the trocar 2500.

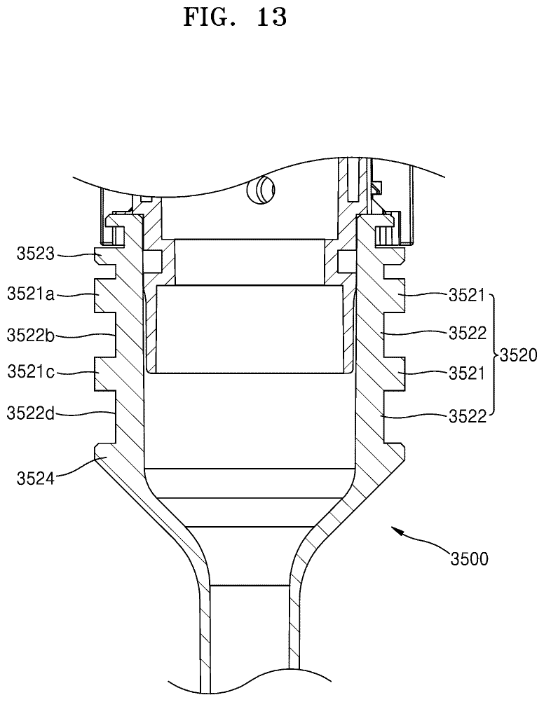

FIG. 13 is a cross-sectional view illustrating a cross section of a trocar 3500 according to another embodiment of the present disclosure.

Referring to FIG. 13, the trocar 3500 according to another embodiment of the present disclosure may have identification portions 3521 formed at positions corresponding to the first switch 2432a and the third switch 2432c, and peripheral portions 3522 formed at positions corresponding to the second switch 2432b and the fourth switch 2432d.

In some embodiments, the trocar 3500 may have a first identification portion 3521a formed at the position corresponding to the first switch 2432a, a first peripheral portion 3522b formed at the position corresponding to the second switch 2432b, a second identification portion 3521c formed at a position corresponding to the third switch 2432c, and a second peripheral portion 3522d formed at a position corresponding to the fourth switch 2432d.

Thus, when the trocar 3500 according to the present embodiment is mounted on the trocar holder 2400, only the first switch 2432a and the third switch 2432c are activated, and the trocar detection unit 2430 may generate a unique signal corresponding to the activation.

When the trocar 3500 according to the present embodiment is mounted on the trocar holder 1400 including the trocar detection unit 1430, which is described with reference to FIG. 10, only the first-1 switch 1432a1, the first-2 switch 1432a2, the third-1 switch 1432c1, and the third-2 switch 1432c2 are activated, and the trocar detection unit 1430 may generate a unique signal corresponding to the activation of the switches in the first and third rows.

As described above, in the trocar detection system 3 according to the embodiments of the present disclosure, the manufacturing process is simpler compared to methods using Hall sensors, which allows manufacturing and maintenance costs to be reduced. The trocar detection system 3 according to the embodiments of the present disclosure may prevent the issue of trocar identification errors by using physical switches.

In some embodiments, the upper boundary portion 1523 of the mounting part 1520 is a boundary portion formed at an upper end of the mounting part 1520, and may distinguish the mounting part 1520 from other parts. For example, the upper boundary portion 1523 may be a protruding region formed on an upper end of the outer surface of the mounting part 1520. The upper boundary portion 1523 may be formed in a band shape protruding along the circumference of the mounting part 1520.

In some embodiments, the lower boundary portion 1524 is a boundary portion formed at a lower end of the mounting part 1520, and may distinguish the mounting part 1520 from other parts. For example, the lower boundary portion 1524 may be a protruding region formed at a lower end of the outer surface of the mounting part 1520. The lower boundary portion 1524 may be formed in a band shape protruding along the circumference of the mounting part 1520.

Here, a height of the mounting part 1520 and a height of the grip part 1421 may be defined by a direction parallel to the longitudinal direction of the trocar 1500. In some embodiments, the height of the mounting part 1520 may correspond to the height of the grip part 1421.

For example, a distance from the upper boundary portion 1523 to the lower boundary portion 1524 in a direction parallel to the central axis C of the trocar 1500 may be defined as the height of the mounting part 1520.

When the height of the grip part 1421 is greater than or equal to the height of the mounting part 1520, the grip parts 1421 may grip the upper boundary portion 1523 and the lower boundary portion 1524 of the mounting part 1520.

When the height of the grip part 1421 is less than the height of the mounting part 1520, the grip parts 1421 may grip a region between the upper boundary portion 1523 and the lower boundary portion 1524 of the mounting part 1520.

In some embodiments, the upper boundary portion 1523 and the lower boundary portion 1524 may guide the grip parts 1421 to grip a certain position of the mounting part 1520.

In some embodiments, when the position at which the trocar 1500 is mounted on the trocar holder 1400 changes each time, an error may occur in the matching between the identification portion 1521 and the switch.

That is, the identification portion 1521 may fail to press the intended switch or may press a different switch.

To minimize such an error, the mounting part 1520 may be provided with a function for guiding the grip position of the grip part 1421.

Referring to FIG. 9, in an embodiment, a height H2 of the grip part 1421 may be approximately equal to a distance H1 between the upper boundary portion 1523 and the lower boundary portion 1524. Accordingly, the region at which the grip parts 1421 grip the mounting part 1520 may remain constant. That is, according to the present embodiment, even when the trocar 1500 is repeatedly mounted on the trocar holder 1400, a deviation in the grip position at which the mounting part 1520 is gripped may be reduced.

In some embodiments, when mounting the trocar 1500 on the trocar holder 1400, the grip parts 1421 may grip a certain region of the mounting part 1520, and the identification portion 1521 may press a specific switch of the trocar detection unit 1430.

According to the present disclosure described above, identification errors in a trocar detection system for a surgical robot can be reduced, and manufacturing costs of the trocar detection system can be lowered.

The present disclosure has been described above with a focus on exemplary embodiments. It will be understood by those skilled in the art that various changes in form and details may be made therein without departing from the essential features of the present disclosure. Therefore, the disclosed embodiments should be considered in descriptive sense only and not for purposes of limitation. The scope of the present disclosure is defined not by the detailed description of the disclosure but by the appended claims, and all differences within the scope will be construed as being included in the present disclosure.

What is claimed is:

1. A trocar detection system for a surgical robot including an arm unit, the trocar detection system comprising:
a trocar holder positioned at one end portion of the arm unit and including a trocar detection unit having a plurality of switches configured to be activated by an external pressure or contact; and
a trocar including a mounting part, which is a region configured to be mounted on the trocar holder,
wherein the mounting part includes an identification portion having a protruding shape so as to be in physical contact with at least one of the plurality of switches when the trocar is mounted on the trocar holder.

2. The trocar detection system of claim 1, wherein the trocar detection unit generates a unique signal corresponding to a combination of activated switches among the plurality of switches, with respect to the trocar mounted on the trocar holder.

3. The trocar detection system of claim 2, further comprising:
a storage part in which trocar type information that matches the unique signal generated by the trocar detection unit is stored; and
a control part configured to determine a type of the trocar based on the trocar type information stored in the storage part and the unique signal transmitted from the trocar detection unit.

4. The trocar detection system of claim 1, wherein the identification portion is formed in a band shape protruding along a circumference of the mounting part.

5. The trocar detection system of claim 1, wherein the identification portion is positioned on an imaginary plane intersecting a central axis of the trocar.

6. The trocar detection system of claim 1, wherein the mounting part has a plurality of identification portions positioned in parallel to each other in a longitudinal direction of the trocar.

7. The trocar detection system of claim 1, wherein
the trocar includes a first type of trocar and a second type of trocar that are different from each other,
the first type of trocar includes an identification portion that is distinguishable from an identification portion of the second type of trocar, and
a combination of switches activated by the identification portion of the first type of trocar is different from a combination of switches activated by the identification portion of the second type of trocar, among the plurality of switches.

8. The trocar detection system of claim 7, wherein the first type of trocar and the second type of trocar are different from each other in at least one of a number, positions, and shapes of the identification portions.

9. The trocar detection system of claim 1, wherein
each of the plurality of switches is positioned within a grid-like section divided into rows and columns, wherein the columns correspond to a direction parallel to a central axis of the trocar while the trocar is mounted on the trocar holder, and the rows correspond to a direction orthogonal to the columns, and
the plurality of switches are positioned in the rows, respectively.

10. The trocar detection system of claim 9, wherein the plurality of switches are positioned in two or more of the columns in one of the rows.

11. The trocar detection system of claim 1, wherein the plurality of switches are pressure-activated.

12. The trocar detection system of claim 1, wherein
the mounting part further includes a peripheral portion that does not protrude relative to the identification portion, and
when the trocar is mounted on the trocar holder, the identification portion presses the plurality of switches, and the peripheral portion does not press the plurality of switches.

13. The trocar detection system of claim 12 wherein, in the mounting part, the identification portion or the peripheral portion is formed at positions respectively corresponding to the plurality of switches.

14. The trocar detection system of claim 1, wherein
the trocar holder further includes a pair of grip parts configured to grip the mounting part, and
the trocar detection unit is positioned between the pair of grip parts.

15. The trocar detection system of claim 14, wherein
a height of the mounting part and a height of each of the pair of grip parts are defined by a direction parallel to a longitudinal direction of the trocar, and
the height of the mounting part corresponds to the height of each of the pair of grip parts.

16. The trocar detection system of claim 14, wherein the mounting part further includes an upper boundary portion and a lower boundary portion,
wherein the upper boundary portion and the lower boundary portion guide the pair of grip parts to grip a certain position of the mounting part.

17. A trocar holder provided on a surgical robot, the trocar holder comprising:
a grip part configured to grip a trocar; and
a trocar detection unit including a plurality of switches configured to be activated when in contact with or pressed by a protruding shape of the trocar,
wherein the trocar detection unit has a combination of activated switches that varies depending on a type of a trocar mounted on the trocar holder.

18. The trocar holder of claim 17, wherein the trocar detection unit generates a unique signal corresponding to the combination of the activated switches among the plurality of switches, with respect to the trocar mounted on the trocar holder.

19. A trocar configured to be mounted on a trocar holder including a trocar detection unit having a plurality of switches, the trocar comprising:
a cannula having a hollow portion formed therein to allow insertion of a surgical instrument through the hollow portion; and
a mounting part disposed at one end portion of the cannula and serving as a region mounted on the trocar holder,
wherein the mounting part includes an identification portion having a protruding shape so as to be in physical contact with at least one of the plurality of switches when the trocar is mounted on the trocar holder.

20. The trocar of claim 19, wherein the identification portion is formed in a band shape protruding along a circumference of the mounting part.
